(19) United States
(12) Patent Application Publication
(10) Pub. No.: US 2024/0115295 A1
(43) Pub. Date: Apr. 11, 2024

(54) MODULAR TULIP ASSEMBLY

(71) Applicant: Astura Medical Inc., Iriving, TX (US)

(72) Inventors: Keith Klausman, Irving, TX (US); Thomas Purcell, Irving, TX (US)

(73) Assignee: Astura Medical Inc., Irving, TX (US)

(21) Appl. No.: 18/378,641

(22) Filed: Oct. 10, 2023

Related U.S. Application Data
(60) Provisional application No. 63/414,834, filed on Oct. 10, 2022.

(51) Int. Cl. A61B 17/70; A61B 17/86

(52) U.S. Cl. CPC A61B 17/7032; A61B 17/8605; A61B 17/8685; A61B 2017/00477

(57) ABSTRACT

A modular tulip assembly is disclosed having a U-shaped tulip body configured to receive a collet bushing, the collet bushing having two stage ramped cut outs configured to a engage flexible wires positioned within the tulip body, the first stage holds the collet bushing in an unlocked state for attachment to a screwhead of a implanted shank, the second stage is a locked state to secure or capture the screwhead by squeezing the screwhead with flexible members of the bushing.

### CROSS-REFERENCE TO RELATED APPLICATIONS

This application claims the benefit of U.S. Provisional Application No. 63/414,834 filed Oct. 10, 2022, which is incorporated herein by reference.

### FIELD

The present invention relates generally to the field of spinal fusion surgery, and more specifically, to a modular tulip assembly that is attached to a screw shank during surgery.

### BACKGROUND

Certain spinal conditions, including a fracture of a vertebra and a herniated disc, indicate treatment by spinal immobilization. Several methods of spinal joint immobilization are known, including surgical fusion and the attachment of pins and bone plates to the affected vertebras. One known device is a stabilization rod interconnecting the two or more pedicle screws to stabilize the vertebras spanned by the screws. The screw includes a channel for receiving the stabilization rod and a nut to apply compressive force between the rod and the screwhead to firmly fix the rod between the spanned vertebras and thus stabilize the spinal vertebrae.

One problem with utilizing conventional pedicle screws is that the tulip/rod saddle can be large and cause limited visibility when using a minimally invasive approach and performing disc preparation.

Another problem is the number of different screws sizes for implant/screw selection in larger cases where a surgeon is implanting 10 plus screws.

Another problem is the difficulty in removing a screw once it is in place.

Lastly, another problem is the high cost and inventory for all the different sizes that are required.

It may be desirable to provide a screw that solves the problems above.

### SUMMARY

The present invention is directed to a modular screw having a tulip/rod saddle that is not assembled to the screw shank prior to the procedure. Implanting the screw shank without the tulip/rod saddle allows maximum visibility when using a minimally invasive approach and performing disc preparation.

The modular tulip/rod saddle allows the surgeon to first implant the screw shanks, then select from the various modular tulip/rod saddle options.

The modular tulip/rod saddle design allows the surgeon to remove and replace the tulip/rod saddle during a procedure without removing the implanted shank.

The modular tulip/rod saddle design reduces cost and screw inventory needed. By utilizing a modular tulip/rod saddle, a set now does not need a tulip for every screw shank, there only needs to be enough tulip options to cover the case.

### BRIEF DESCRIPTION OF THE DRAWINGS

FIG. 1 is a perspective exploded view of a modular tulip assembly 100 for use with a pedicle screw shank.

FIG. 2 shows one embodiment of a collet bushing.

FIG. 3 is a sectional perspective view of the inner portion of the tulip.

FIG. 4 is a sectional perspective view of the inner portion of the modular tulip assembly.

FIG. 5 is a sectional top view of the modular tulip assembly.

FIGS. 6A and 6B are a side view and a sectional view of the modular tulip assembly in the unlocked state.

FIGS. 7A and 7B are a side view and a sectional view of the modular tulip assembly in the locked state.

FIG. 8 is a side view showing having a screw shank of a pedicle screw that has been implanted.

FIGS. 9A, 9B are a side view and a sectional side view showing placement of the modular tulip assembly on the screw.

FIGS. 10A, 10B are a side view and a sectional side view showing the screwhead in the locked state within the modular tulip assembly.

FIG. 11 is a perspective view of the collet busing.

FIG. 12 is a perspective sectional view of the modular tulip assembly in the secured or captured state.

FIG. 13 is a sectional view of the screwhead locked within the collet busing in the secured or captured state.

FIG. 14 is a sectional view of the screwhead within the collet busing in the unlocked state.

### DETAILED DESCRIPTION

A modular tulip assembly is disclosed having a U-shaped tulip body configured to receive a collet bushing, the collet bushing having two stage ramped cut outs configured to engage flexible wires positioned within the tulip body. The two stage ramped cut outs act like a ratcheting mechanism that allows motion in one direction and prevents motion in a second direction. The flexible wires in the first stage engage lower cut outs to hold the collet bushing in an unlocked state for attachment to a screwhead of an implanted shank. The flexible wires in the second stage engage upper cut outs secure the screwhead in a locked state to secure or capture the screwhead by squeezing the screwhead with flexible members of the bushing.

In some embodiments, the flexible wires provide an audible sound and tactile feedback to the surgeon when transitioning the collet bushing from the unlocked state to the locked state. Feedback gives the surgeon confidence that the change of state is completed successfully. The flexible wires within the two stage ramped cut outs also stop the collet bushings from going back into the unlocked state which eliminates the possibility of the tulip removing from the screwhead accidentally. A removal tool may be used to disengage the flexible wires to bring the tulip back to the unlocked state from the locked state for removal. A removal tool provides a dedicated process for removal.

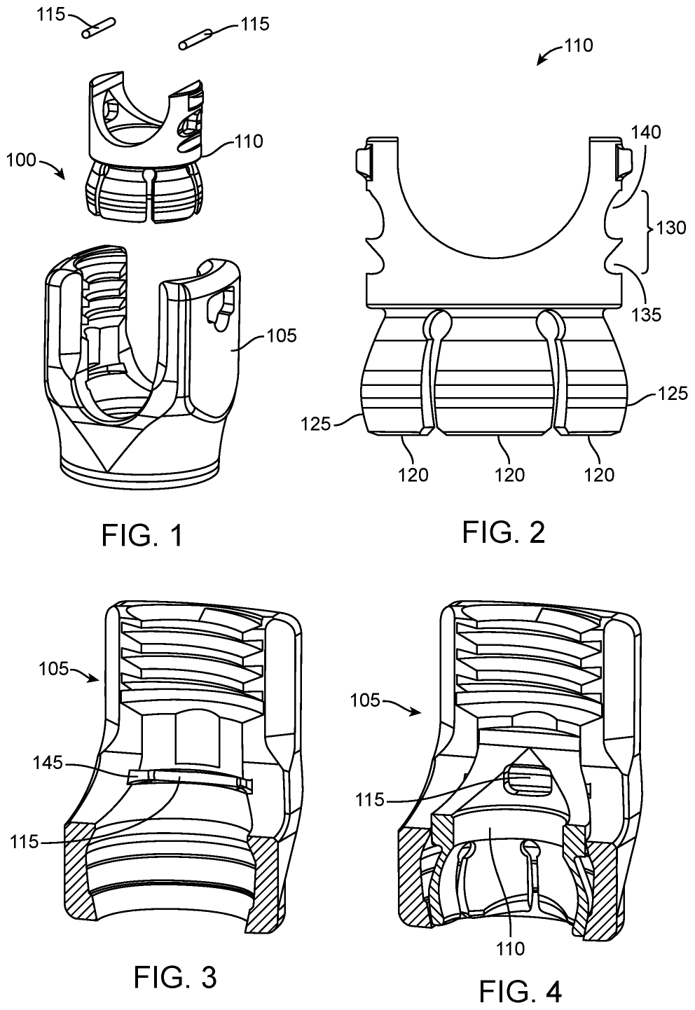

FIG. 1 is a perspective exploded view of a modular tulip assembly 100 for use with a pedicle screw shank comprising a U-shaped tulip body 105, a collet bushing 110, and flexible wires 115, such as nitinol wires. The U-shaped tulip and collet bushing are configured to hold a rod R. The interior of the tulip body 105 includes a threaded portion to receive a rod locking device, such as a set screw S. The set screw S is configured to reduce the rod R into the U-shaped tulip 105 and push the rod R 300 against the lower portion to lock the rod in the tulip 110.

FIG. 2 shows one embodiment of a collet bushing 110 having flexible members 120 for receiving a screwhead with a tapered distal external profile 125 for taper locking against the inside of the tulip 105 and squeezing onto the screwhead. The collet bushing 110 also includes two stage cut outs 130 on both sides that interact with the flexible wire 115. The first stage is a lower cut out 135 and the second stage is an upper cut out 140. In other embodiments may include one or more cutouts, a one stage cutout or multistage cutouts.

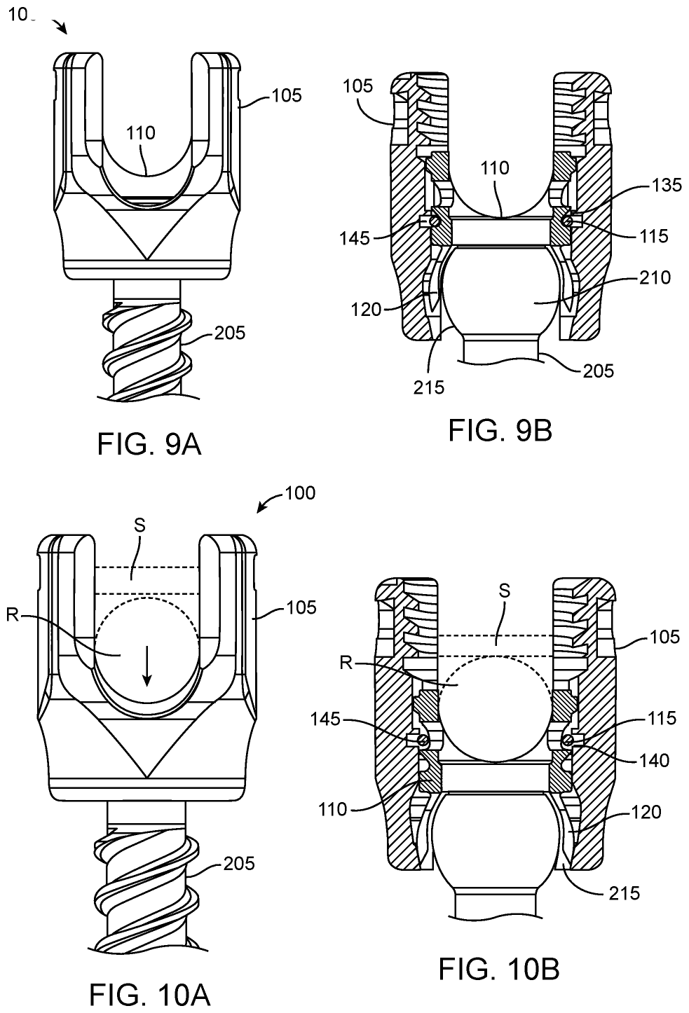

The first stage lower cut out 135 initially engages the flexible wire 115 when the collet bushing 110 translates downard in the tulip 105. The collet bushing 110 is configured to translate downward inside the tulip 105 to contact and flex the flexible wires 115 into their radial grooves in the wall of the tulip 105. At this time the flexible wires 115 engage the lower cut outs 135 and spring back straight and sit at the cut outs 135 of the collet bushing 110 which stops the collet bushing 110 from translating upwards. The lower cut out 135 includes a lower portion that is shaped to engage the flexible wire 115 to prevent upward translation of the collet bushing 110, and an upper ramped or tapered portion that is shaped to engage and compress the flexible wire 115 into the grooved slot 145 to allow continued downward translation of the collet bushing 110. The lower portion may be U-shaped or saddle shaped having a lower center area for the flexible wire to sit within. In the first stage the collet bushing 110 is in an unlocked state (see FIG. 9B).

The second stage upper cut out 140 engages the flexible wire 115 that has been compressed by the ramped or tapered portion in stage one as the collet bushing 110 continues to translate downward in the tulip 105. At this time the flexible wires 115 engage the upper cut outs 140 and spring back straight into the upper cut outs 140. The upper cut out 140 includes a lower portion that is shaped to engage the flexible wire 115 to prevent upward translation of the collet bushing 110. In the second stage upper cut out 140 the collet bushing 110 is in a locked state to secure and lock the screwhead in the flexible members 120 of the bushing 110 (see FIG. 10B).

The addition of nitinol wires provides an audible sound and tactile feedback when transitioning the collet bushing from the Unlocked state to the locked state. Feedback gives the surgeon confidence that the change of state was completed successfully. The nitinol wires also stop the collet bushing from going back into the unlocked state which eliminates the possibility of the tulip removing from the screwhead accidentally. The addition of nitinol wires also requires a removal tool to bring the tulip back to the Unlocked state for removal. The requirement of a removal tool provides a dedicated process for removal.

FIG. 3 is a sectional perspective view of the inner portion of the tulip 105 showing the flexible wire 115 positioned within radial grooves 145. The radial grooves 145 are shaped to fit the flexible wire 115 when they are flexed radially to allow passaged of the collet bushing 110.

FIG. 4 is a sectional perspective view of the inner portion of the modular tulip assembly 100 showing the collet bushing 110 engaging the flexible wire 115 within the tulip 105.

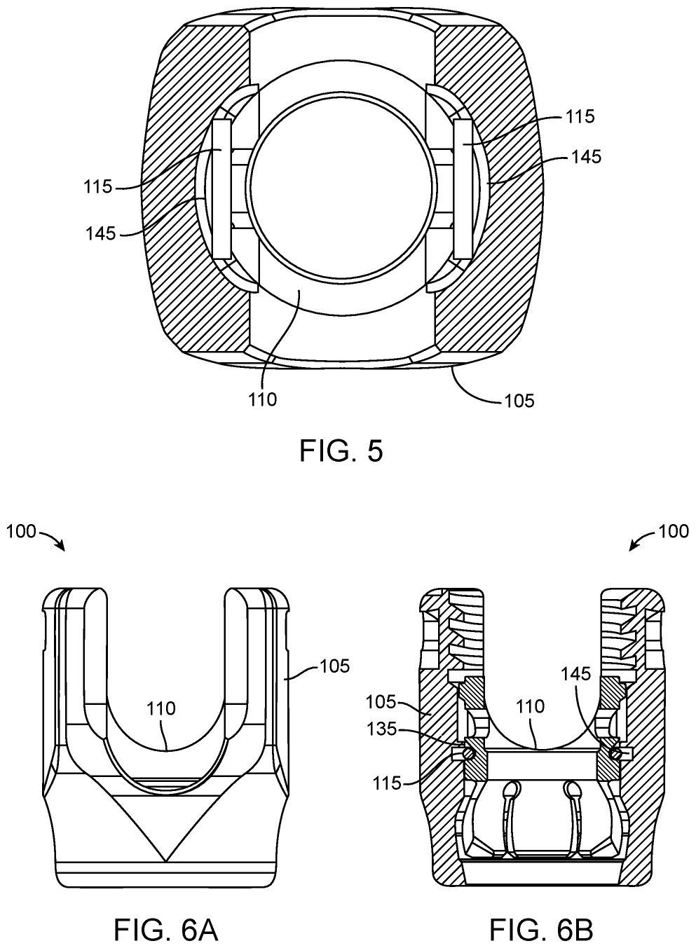

FIG. 5 is a sectional top view of the modular tulip assembly 100 showing the collet bushing 110 engaging the flexible wire 115 within the tulip 105. In this view, the flexilbe wires are within the first stage cut out 135 or the second stage cut out 140. Also shown is the groove slot 145 that flexible wire 115 is compressed into to allow downward translation of the collot bushing 110 in the tulip 105.

FIGS. 6A and 6B are a side view and a sectional view of the modular tulip assembly 100 in the unlocked state. The collet bushing 110 is inserted into a central opening of the tulip 105 and is translated downward, compressing the flexible wire 115 into the groove slot 145 until it reaches the first stage lower cut out 135, then the collet bushing 110 is in an unlocked state. In the unlocked state, the modular tulip assembly 100 is pushed on a screwhead of a screw that has been implanted.

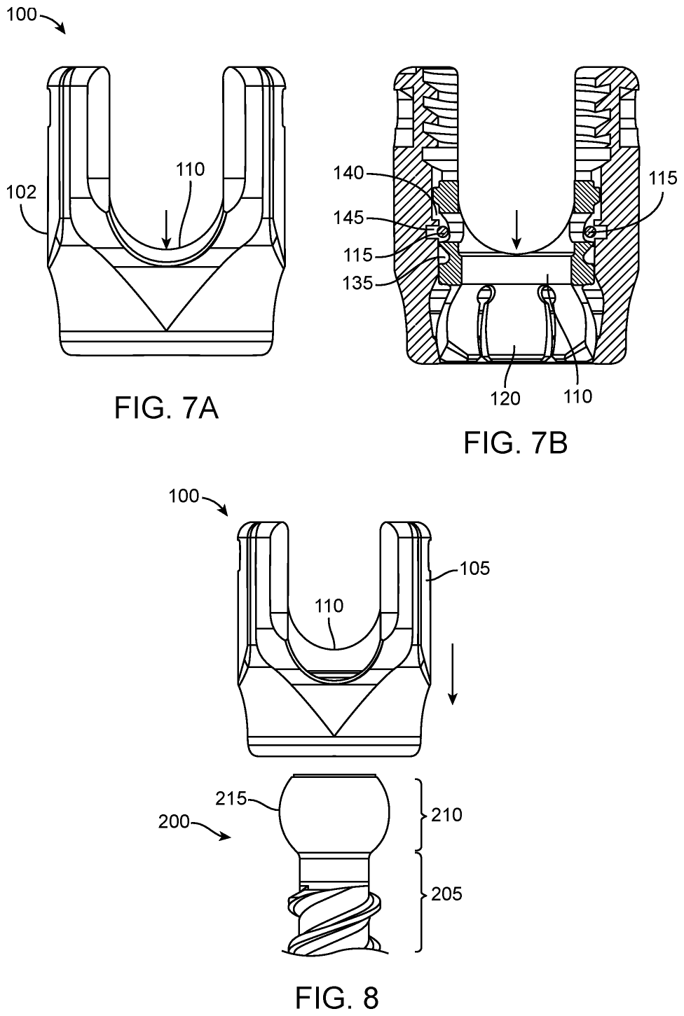

FIGS. 7A and 7B are a side view and a sectional view of the modular tulip assembly 100 in the locked state. Once the screwhead is positioned within the flexible members 120 the collet bushing 110 is translated downward and the tapered distal external profile 125 of the flexible members 120 engage the tapered lower profile of the tulip 105. During this downward translation, the ramped portion of the lower cut out 135 compresses the flexible wire 115 into the groove slot 145 until it reaches the second stage upper cut out 140, then the flexible wire 115 enters the second stage upper cut out 140, locking the collet bushing 110 in the secured or captured state.

FIG. 8 is a side view showing having a screw shank 205 of a pedicle screw 200 that has been implated. The screw shank 205 is implanted without the modular tulip assembly 100 which allows maximum visibility when using a minimally invasive approach. The proximal end of the screw 200 includes a screwhead 210 having a curved outer surface 215.

Once the screw shank 205 has been implanted, the surgeon selects a modular tulip assembly 100 from the various modular tulip assembly 100 options. The unlocked modular tulip assembly 100 is in the unlocked state.

FIGS. 9A, 9B are a side view and a sectional side view showing placement of the modular tulip assembly 100 on the screw 200 in the unlocked state with the flexible wires 115 positioned in the lower cut out 135 of the collet bushing 110. The modular tulip assembly 100 includes a bottom opening that is sized to receive the screwhead 210. When the modular tulip assembly 100 is pushed onto the screw 200, the screwhead 210 is postioned within the flexible members 120 of the collet bushing 110. In the inlocked state, the modular tulip assembly 100 may be rotated or tilted to the desired orientation.

FIGS. 10A, 10B are a side view and a sectional side view showing the screwhead 210 in the locked state within the modular tulip assembly 100. After placement and positioning the modular tulip assembly 100 in the disired orientation on the screwhead 210, the collet bushing 110 is translated downward with the flexible members 120 squeezing onto the curved surface 215 of the screwhead 210 as the tapered distal external profile 125 engages a tapered portion inside of the tulip 105 creating a taper locking against the screwhead 210 by the flexible members 120. At the same time, the flexible wire 115 translates from the lower cut out 135 to the upper cut out 140, locking the bushing in place.

When the modular tulip assembly 100 is in the secured or captured state, a spinal rod R may be placed in the tulip/collet bushing opening and secured with a setscrew S.

The modular tulip assembly 100 includes a bolttom opening that is sized to receive the screwhead 210. When the modular tulip assembly 100 is pushed onto the screw 200, the screwhead 210 is postioned within the flexible members 220 of the collet bushing 210. In the inlocked state, the modular tulip assembly 100 may be rotated or tilted to the desired orientation.

Having a modular tulip assembly 100 allows for the surgeon to first implant the screw shanks, then select from the various modular modular tulip assembly 100 options.

During assembly of the modular tulip assembly 100, the collet busing 110 is inserted into the tulip 105 and a downward force on the collet bushing 120 within the tulip 105 flexes the flexible wires 115 into radial grooves 145 on the inner wall of the tulip 105 allowing the collet bushing 110 to translate downward inside the tulip 105 until flexible wires 115 engage the lower unlocked cut out 135 and spring back straight into the lower cut out 135. The collet bushing is now in the unlocked state.

In the unlocked state, the flexible members 120 are configued to flex open when pushed onto the screwhead 210 to mount the modular flexible tulip assembly 100. Once the scew head is within the flexible members 120, the collet bushing 110 is translated downward until the tapered distal external profile 125 engages the lower surface the inside of the tulip 105 and squeeze the screwhead 210 to taper lock the screw 200 within the collett bushing 110. During this second downward translation, the shape of the lower unlocked cut out 135 will flex the flexible wires 115 into the radial grooves 145 until the wires 115 engage the upper cut out 140 and spring back straight into the upper cut out 140 to lock the collet bushing 110 in place and prevent it from translating uoward.

The modular tulip assembly 100 is configured to be removeable from the screw 200 after being locked in the secured or captured state. This is achieved by moving the nitinol wires 115 out of the upper cut outs 140 and into the raidal grooves 145, then pulling the collet bushing 110 upward until the flexible wires 115 are back in the lower cut outs 135 in the unlocked state by releasing the screwhead 210 from the taper lock with the flexible members 220. The tulip 105 can then be pulled off the screwhead 210.

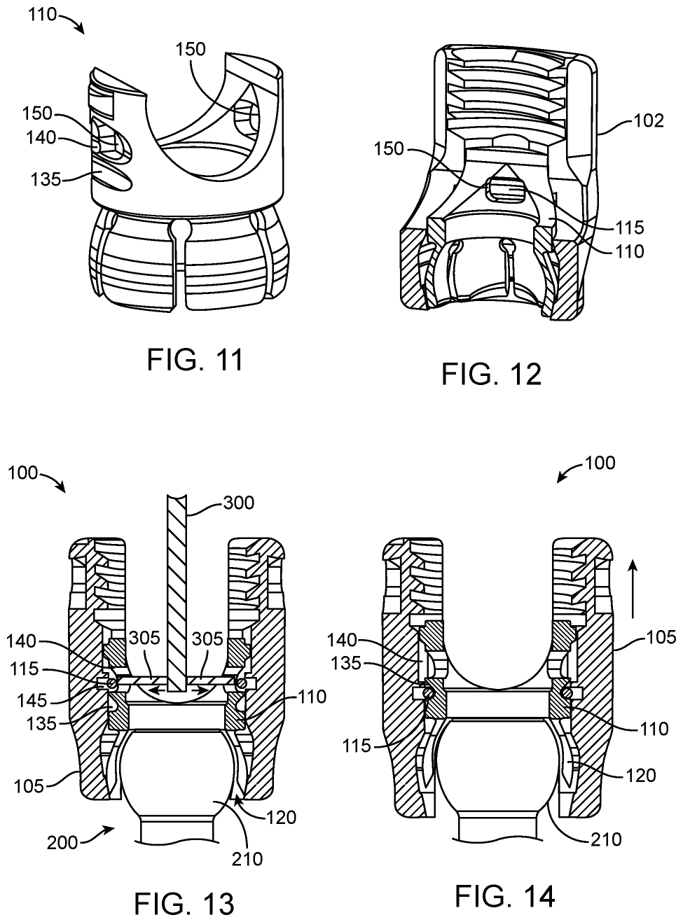

FIG. 11 is a perspective view of the collet busing 110 showing an opening or hole 150 through the wall at upper cut out 140. The hole 150 allows access to the flexible wire 115 when the collet bushing 110 is positioned within the tulip 102.

FIG. 12 is a perspective sectional view of the modular tulip assembly 100 in the locked state with the flexible wire 115 positioned in the upper cut out 140. The hole 150 allows contact with the flexible wire 110.

FIG. 13 is a sectional view of the screwhead 210 locked within the collet busing 110 in the secured or captured state. A removal instrument 300 is inserted into the tulip 105 having wire push arms 305 configured to extend through the openings to push the flexible wire 115 into the radial grooves 145 to unlock the modular tulip assembly 100.

FIG. 14 is a sectional view of the screwhead 210 within the collet busing 110 in the unlocked state. The modular tulip assembly 100 may be removed from the screw 200.

The addition of the flexible wires provides an audible sound and tactile feedback when transitioning the collet bushing from the Unlocked state to locked state. Feedback gives the surgeon confidence that the change of state is completed successfully. The nitinol wires also stop the collet bushing from going back into the unlocked state which eliminates the possibility of the tulip removing from the screwhead accidentally. The addition of the flexible wires may also require a removal tool to bring the tulip back to the unlocked state for removal. The requirement of a removal tool provides a dedicated process for removal.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A modular tulip assembly comprising:
a collet bushing having flexible members configured to receive a screwhead and two stage ramped cutouts on an outer surface;
a modular tulip configured to receive the collet bushing; and
one or more flexible wires within the modular tulip configured to engage the two stage ramped cutouts to lock the collet bushing in a first stage unlocked state and a second stage locked state.

2. The modular tulip assembly of claim 1, wherein in the unlocked state, the modular tulip assembly is configured to be pushed on a screwhead of a screw.

3. The modular tulip assembly of claim 1, wherein the locked state the collet bushing is translated downward in the modular tulip and the flexible members engage a tapered lower profile of the tulip.

4. The modular tulip assembly of claim 1, wherein the tulip includes a bottom opening for a threaded shaft of the screw to go through.

5. The modular tulip assembly of claim 1, wherein the tulip is a U-shaped tulip having side walls with U-shaped openings sized to receive a rod.

6. The modular tulip assembly of claim 5, wherein the interior of the U-shaped opening of the U-shaped tulip may be threaded to accept a set screw configured to be tightened and torqued into the rod, which contacts the collet bushing, which squeezes the screwhead to lock.

7. A modular tulip assembly comprising:
a collet bushing having an outer surface with two stage ramped cutouts and flexible members configured to receive a pedicle screwhead;
a modular tulip having an inner wall with radial grooves configured to slidingly receive the collet bushing; and
one or more flexible wires positioned within the modular tulip proximate the radial grooves configured to engage the two stage ramped cutouts as the collet bushing translate downward in the modular tulip.

8. The modular tulip assembly of claim 7, wherein the two stage ramped cutouts include a first stage lower cut out with the flexible members in an unlocked state and a second stage upper cut out with the flexible members in a locked state.

9. The modular tulip assembly of claim 8, wherein in the unlocked state, the flexible members are configured to be pushed on the pedicle screwhead.

10. The modular tulip assembly of claim 8, wherein in the locked state the flexible members squeeze and lock the pedicle screwhead.

11. The modular tulip assembly of claim 8, wherein the flexible members include a tapered distal external profile configured to engage a tapered lower profile of the modular tulip during downward translation, moving the flexible members from the unlocked state to the locked state.

12. The modular tulip assembly of claim 7, wherein the two stage ramped cutouts include a ramped or tapered portion configured to compress the flexible wires during downward translation of the collet bushing and a lower u-shaped or saddle shaped portion with a lower center area for the flexible wires to sit in.

13. The modular tulip assembly of claim 12, wherein the lower U-shaped or saddle shaped portion is configured to prevent upward translation of the collet bushing.

14. The modular tulip assembly of claim 7, wherein the modular tulip includes a U-shaped opening sized to receive a rod.

15. The modular tulip assembly of claim 14, wherein the interior of the modular tulip U-shaped opening may be threaded to accept a set screw configured to be tightened and torqued into the rod, which contacts the collet bushing to translate downward and the flexible members squeeze the pedicle screwhead to lock.

16. A modular tulip assembly comprising:
a collet bushing having distal flexible members configured to receive a screwhead and two stage ramped cutouts on an outer surface;
a modular tulip having a U-shaped tulip body an inner wall with radial grooves configured to slidingly receive the collet bushing; and
one or more flexible wires within the modular tulip configured to engage the two stage ramped cutouts;
wherein the two stage ramped cutouts include a first stage lower cut out in an unlocked state with the flexible members configured to be pushed on the screwhead, and a second stage upper cut out in a locked state with the flexible members configured to squeeze and lock the screwhead.

17. The modular tulip assembly of claim 16, wherein the two stage ramped cutouts include a ramped or tapered portion configured to compress the flexible wires into the radial grooves during downward translation of the collet bushing, and a lower u-shaped or saddle shaped portion with a lower center area for the flexible wires to sit in, the lower u-shaped or saddle shaped portion is configured to prevent upward translation bushing.

18. The modular tulip assembly of claim 16, wherein the interior of the U-shaped body may be threaded to accept a set screw configured to be tightened and torqued into the rod, which contacts the collet bushing, which squeezes the of the collet screwhead to lock.

19. The modular tulip assembly of claim 16, wherein the flexible members include a tapered distal external profile configured to engage a tapered lower profile of the modular tulip during downward translation, moving the flexible members from the unlocked state to the locked state.

20. The modular tulip assembly of claim 16, wherein the flexible wires are configured to make an audible click and/or tactile feedback when transitioning the collet bushing from the unlocked state to the locked state.
